(19) United States
(12) Patent Application Publication
(10) Pub. No.: US 2022/0025129 A1
(43) Pub. Date: Jan. 27, 2022

(54) LACTIDE CONTAINING POLYESTER-POLYETHYLENE GLYCOL TRIBLOCK THERMORESPONSIVE COPOLYMERS

(71) Applicants: Abraham J. DOMB, Jerusalem (IL); Noam STEINMAN, Jerusalem (IL)

(72) Inventors: Abraham J. DOMB, Jerusalem (IL); Noam STEINMAN, Jerusalem (IL)

(73) Assignee: Yissum Research Development Company of the Hebrew University of Jerusalem Ltd., Jerusalem (IL)

(21) Appl. No.: 17/294,086

(22) PCT Filed: Nov. 14, 2019
(86) PCT No.: PCT/IL2019/051242

§ 371 Date: May 14, 2021

Related U.S. Application Data
(60) Provisional application No. 62/767,748, filed on Nov. 15, 2018.

(51) Int. Cl. C08G 81/00; C08G 63/08; C08L 71/02; A61K 47/34; A61K 9/00; A61K 8/90; A61Q 19/00

(52) U.S. Cl. CPC C08G 81/00; C08G 63/08; C08L 71/02; A61K 47/34; C08L 2201/06; A61K 8/90; A61Q 19/00; A61K 2800/10; A61K 9/0024

(57) ABSTRACT

The inventors of the technology disclosed herein have developed triblock copolymers of lactide-containing polyesters and poly(ethylene glycol), PEG, having thermoresponsive properties.

### TECHNOLOGICAL FIELD

The invention generally concerns compositions comprising lactide containing polyester-polyethylene glycol triblocks such as PLGA-PEG-PLGA and uses thereof.

### BACKGROUND

Effective controlled drug release provides the advantage of sustained therapeutic activity over a long time period. Poly(lactic-co-glycolic acid) (PLGA) has been studied extensively in this field due to its excellent biocompatibility and flexibility in terms of chemical composition. The potential to inject biodegradable polymers directly to tissues provides an attractive platform for the delivery of therapeutic materials to the tissues without the need to remove unwanted by-products.

Polymeric hydrogels are three-dimensional systems that are able to absorb large amounts of water due to physical crosslinking of the polymer. ‘Smart’ hydrogels have been developed to undergo a sol-gel transition as a response to a variety of external stimuli, namely pH and temperature. In these systems, gelation is contingent upon the external stimulus; without such stimulus the polymer merely dissolves in the aqueous medium. This feature has allowed for the polymer to be injected to a site where the prescribed stimulus may be found, thereby causing the gel to form in situ.

### GENERAL DESCRIPTION

Poly(D,L-lactic acid-co-glycolic acid)-b-poly(ethylene glycol)-b-poly (D,L-lactic acid-co-glycolic acid) (PLGA-PEG-PLGA) triblock copolymers have been widely used to make safe, biocompatible, biodegradable and crucially FDA-approved thermoresponsive hydrogels. The sol-gel transition temperature is affected by concentration, chain length of PEG, PLGA, the ratio between them, as well as the lactic acid/glycolic acid (LA:GA) ratio within the PLGA blocks. While extensive research has been done on various applications of PLGA-PEG-PLGA triblock copolymers, including preparation of stimuli-responsive micelles, controlled release of proteins, or model small molecule drugs, and local delivery for bone regeneration, little attention has been given to the role of block chain molecular weight (MW) in determining the gelling temperature of the thermoresponsive polymer gels.

The inventors of the technology disclosed herein have discovered that triblock copolymers of lactide-containing polyesters and poly(ethylene glycol), PEG, (herein ‘triblock’) form a thermoresponsive polymer, e.g., which may be in a form of a hydrogel aqueous solution, wherein in the triblock copolymer, the PEG is of a molecular weight between 1,000 and 3,000 Da. In one of the herein exemplified cases, triblocks such as PLGA-PEG-PLGA have been prepared and used as thermoresponsive materials at these PEG molecular weights and material ratio compositions.

In the specific case, the inventors have determined that by using a PEG of a molecular weight between 1,000 and 3,000 Da, at a PLGA/PEG ratio of between 1 and 4, or a lactide:glycolide (LA:GA) ratio of about 6, PLGA-PEG-PLGA compositions can be obtained to have a gelation transition temperature between 10° C. and 50° C., or between room temperature (23° C.) and 50° C. In other words, by modifying the MW of PEG, by modifying the PLGA:PEG ratio or by modifying the LA:GA ratio, gelation of the PLGA-PEG-PLGA triblock may be obtained at a temperature ranging between 10° C. and 50° C.

The polymers of the invention have a T_{gel} greater than 10° C.

As demonstrated herein, at different poly(ethylene glycol) (PEG)-based polymers or with PEGs of higher MWs, gellous triblocks could not be formed.

The lactide-containing polyesters are segment polymers which include any lactide groups such as D,L-PLA (D,L-lactide or poly(D,L-lactic acid)), L-PLA (L-lactide or poly(L-lactic acid)), D-PLA (D-lactide or poly(D-lactic acid)), PLGA (poly(D,L-lactic acid-co-glycolic acid)), PCL (polycaprolactone) and others. Together with PEG, these lactide segments form the triblock copolymer of the invention.

Triblock copolymers of lactide-containing polyesters and PEG may thus include, for example and without limitation, the following D,L-PLA-PEG-D,L-PLA, D-PLA-PEG-D-PLA, L-PLA-PEG-L-PLA, PLGA-PEG-PLGA, PCL-PEG-PCL, in triblock copolymer forms. Hybrid or mixed triblocks are also within the scope of the present invention, wherein the triblock comprises two different lactide polyester segments. In such embodiments, triblock copolymers of the following structures may be considered to include D,L-PLA-PEG-L-PLA, D,L-PLA-PEG-D-PLA, D-PLA-PEG-L-PLA, L-PLA-PEG-PLGA, D-PLA-PEG-PLGA, PLGA-PEG-PCL, PCL-PEG-D,L-PLA and others.

Thus, the invention provides a triblock copolymer constructed of a lactide-containing polyester and poly(ethylene glycol), PEG, wherein in the triblock copolymer, the PEG is of a molecular weight between 1,000 and 3,000 Da.

In some embodiments, the lactide-containing polyester is selected from D,L-PLA, L-PLA, D-PLA, PLGA and PCL.

In some embodiments, the triblock copolymer is of a structure selected from D,L-PLA-PEG-D,L-PLA, D-PLA-PEG-D-PLA, L-PLA-PEG-L-PLA, PLGA-PEG-PLGA and PCL-PEG-PCL.

In some embodiments, the triblock copolymer is selected from hybrid triblocks containing PEG and one lactide-containing polyester segment selected as above.

In some embodiments, the hybrid triblock is selected from D,L-PLA-PEG-L-PLA, D,L-PLA-PEG-D-PLA, D-PLA-PEG-L-PLA, L-PLA-PEG-PLGA, D-PLA-PEG-PLGA, PLGA-PEG-PCL and PCL-PEG-D,L-PLA.

In some embodiments, the lactide-containing polyester segment is PLGA. In some embodiments, the triblock is PLGA-PEG-PLGA, namely poly(D,L-lactic acid-co-glycolic acid)-b-poly(ethylene glycol)-b-poly (D,L-lactic acid-co-glycolic acid).

In some embodiments, the lactide-containing polyester segment is D,L-PLA. In some embodiments, the triblock is D,L-PLA-PEG-D,L-PLA, namely poly(D,L-lactic acid)-poly(ethylene glycol)-poly(D,L-lactic acid).

The invention thus provides a PLGA-PEG-PLGA triblock copolymer, wherein in the PLGA-PEG-PLGA triblock copolymer, the PEG is of a molecular weight between 1,000 and 3,000 Da, the PLGA and PEG being present at a ratio of between 1 and 4, and wherein the triblock copolymer having a gelation temperature between 10 and 50° C.

The invention further provides a PLGA-PEG-PLGA triblock copolymer, wherein in the PLGA-PEG-PLGA triblock copolymer, the PEG is of a molecular weight between 1,000 and 3,000 Da, one or both of the PLGA segments being constructed of lactide and glycolide (LA:GA) moieties at a ratio of about 6, and wherein the triblock copolymer having a gelation temperature between 10 and 50° C.

The invention further provides a PLGA-PEG-PLGA triblock, characterized by:
in PLGA-PEG-PLGA triblock copolymer, the PEG is of a molecular weight between 1,000 and 3,000 Da,
in the PLGA-PEG-PLGA triblock copolymer, the PLGA and PEG being present at a ratio of between 1 and 4,
in the PLGA-PEG-PLGA triblock copolymer, one or both of the PLGA segments is constructed of lactide and glycolide (LA:GA) moieties at a ratio around about 6; and
the triblock copolymer having a gelation temperature between 10 and 50° C.

The invention further provides a material having a structure PLGA-PEG-PLGA, the material being any one or more of those entered (listed) in Table 1 (any one of those designated as entries 1 through 26).

The invention further contemplates a method of manufacturing a PLGA-PEG-PLGA triblock copolymer having a gelation temperature between 10 and 50° C., the method comprising reacting PEG of a molecular weight between 1,000 and 3,000 Da with D,L-lactic acid (LA) and a glycolide (GA) at (1) a LA:GA ratio of about 6; and/or (2) with a D,L-lactic acid (LA) and a glycolide (GA) amounts sufficient to achieve a PLGA/PEG ratio of between 1 and 4; under conditions permitting formation of the triblock copolymer.

The conditions permitting formation of the triblock copolymer are those permitting ring-opening polymerization (ROP) of poly(ethylene glycol) (PEG), e.g., with D,L-lactide and glycolide. In some embodiments, ROP is achieved in the presence of a catalyst. Such catalysts may be selected amongst stannous catalysis, e.g., stannous octoate.

In some embodiments, ROP conditions include thermal treatment of the ingredients in the presence of the catalyst at a temperature above room temperature. In some embodiments, the temperature is between 75 and 200° C. or between 100 and 200° C. or between 100 and 150° C. or between 120 and 150° C. In some embodiments, ROP is achievable in an organic solvent having a high boiling point and the reaction mixture is heated to the boiling point of the organic solvent.

In some embodiments, the triblock is a triblock shown in FIG. 2, wherein X=22-69; Y=7-56; Z=0-28 where Z<Y/2.

As used herein, the PLGA-PEG-PLGA triblock copolymer refers to poly(D,L-lactic acid-co-glycolic acid)-b-poly(ethylene glycol)-b-poly (D,L-lactic acid-co-glycolic acid) triblock copolymer. The triblock copolymer is thus a polymer comprising one PEG polymer segment and two lactide, e.g., PLGA, segments, wherein the PEG segment is positioned at the canter of the triblock. Triblocks according to the invention, i.e., defined by PEG molecular weight, lactide:PEG ratio, e.g., PLGA:PEG ratio or LA:GA ratio, may be prepared following any synthetic methodology known in the art. The molecular weight (MW) of PEG is selected to be between 1,000 and 3,000 Da; the MW of the lactide segment, e.g., PLGA or precursors thereof may be selected to achieve a lactide:PEG, e.g., PLGA:PEG, ratio between 1 and 4. Thus, the final MW of PLGA may be selected based on the MW of PEG used and the MW of the triblock may thus vary. In some embodiments, the invention provides a polymeric material comprising a triblock copolymer as defined. In other embodiments, the invention provides a material consisting the triblock polymer as defined.

The PEG molecular weight in a triblock of the invention is said to be between 1,000 and 3,000 Da. In other words, the PEG segment in a triblock of the invention is of a MW between 1,000 and 3,000 Da. The MW may be selected from between 1,000 and 1,100, 1,000 and 1,200, 1,000 and 1,300, 1,000 and 1,400, 1,000 and 1,500, 1,000 and 1,600, 1,000 and 1,700, 1,000 and 1,800, 1,000 and 1,900, 1,000 and 2,000, 1,000 and 2,100, 1,000 and 2,200, 1,000 and 2,300, 1,000 and 2,400, 1,000 and 2,500, 1,000 and 2,600, 1,000 and 2,700, 1,000 and 2,800, 1,000 and 2,900, 1,500 and 1,600, 1,500 and 1,700, 1,500 and 1,800, 1,500 and 1,900, 1,500 and 2,000, 1,500 and 2,100, 1,500 and 2,200, 1,500 and 2,300, 1,500 and 2,400, 1,500 and 2,500, 1,500 and 2,600, 1,500 and 2,700, 1,500 and 2,800 or between 1,500 and 2,900 Da.

In some embodiments, the PEG molecular weight in a triblock of the invention is not greater than about 3,000 Da. As the accurate molecular weight may be difficult to determine for each segment in every polymeric material, the expression ‘about 3,000’ should mean not greater than 3,000 Da plus between 1 and 10%. In other words, the maximum MW is 3,000+10%, namely up to 3,300 Da. Excluded from triblocks and compositions of the invention are PLGA-PEG-PLGA triblocks wherein the PEG is of a molecular weight of 3,300 Da and higher.

Where PLGA is the lactide of choice, the PLGA:PEG ratio is a MW ratio of between 1 and 4. That means that based on the selected MW of PEG, the MW of the PLGA in the triblock is determined and selected. The ratio may be 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.7, 3.8, 3.9 or 4.

In some embodiments, the PLGA:PEG ratio is 1.08, 1.19, 1.20, 1.25, 1.32, 1.34, 1.36, 1.37, 1.53, 1.58, 1.61, 1.66, 1.88, 1.89, 1.91, 1.95, 1.99, 2, 2.01, 2.12, 2.16, 2.18, 2.21, 2.34, 2.47 or 3.02.

As indicated hereinbelow, the PLGA-PEG-PLGA triblock is prepared by polymerization of poly(ethylene glycol) (PEG) with D,L-lactide (LA) and glycolide (GA). In some embodiments, the triblocks of the invention are prepared by selecting a LA:GA MW ratio to be around or about 6. The expression ‘around 6’ or ‘about 6’ means a ratio of 6±10%. Thus, a ratio of about 6 means a ratio between 5.4 and 6.6. In some embodiments, the ratio is 5.4, 5.5, 5.6, 5.7, 5.8, 5.9, 6, 6.1, 6.2, 6.3, 6.4, 6.5 or 6.6. In some embodiments, the ratio is between 5.4 and 6 or between 6 and 6.6.

As exemplified, both the PLGA:PEG ratio, as well as the LA:GA ratio may be determined experimentally by NMR or by other spectroscopic or quantitative methods.

Triblocks of the invention are characterized by having a gelation temperature between about 10 and 50° C. This means that the triblocks of the invention exhibit a liquid state at a temperature suitable for application to a subject or for a utility in other non-medicinal uses. In some embodiments, the triblocks are liquids at, for example, at room temperature and undergo gelation at a physiological temperature (between 30 and 40° C.), for example, at about 32° C. for skin surface temperature, or about 40° C. for a sick person.

Triblocks of the invention may be used neat or may be formed into compositions or formulations comprising at least one triblock, as defined, and a carrier. In some embodiments, the composition or formulation is an aqueous formulation comprising water as a carrier. In some embodiments, the carrier is an aqueous gel. The amount of the PLGA-PEG-PLGA triblock in a formulation may be varied based on the particular use. In some embodiments, an aqueous formulation comprises between 10 and 30% (W/V) of the at least one triblock. Thus, the invention further provides compositions/formulations comprising at least one triblock, as defined herein, and a carrier.

Compositions or formulations of the invention may comprise one or more than one triblock. In cases where two or more triblocks are used, each may be different in at least one or more of PEG molecular weight, lactide segment, lactide molecular weight, PLGA molecular weight, PLGA:PEG ratio, a different gelation temperature, and others. In some embodiments, the two or more different triblocks may be selected to have different gelation temperatures while having liquid phases at room temperature, or at temperatures below room temperature (but higher than 10° C.). In such embodiments, while the combination of the two or more triblocks may be delivered in a single composition or formulation, each of the triblock will undergo gelation at a different temperature. Conversely, two or more triblocks may be selected to exhibit liquid phases at different temperatures but will have a similar gelation temperature.

Typically, compositions or formulations of the invention may be used as hydrogels, as matrix materials or as scaffold materials for carrying and delivering an active or a non-active material to a target site. The choice of carrier will be determined in part by the particular active or non-active agent, as well as by the particular method used to deliver or administer the composition or formulation. Accordingly, there is a wide variety of suitable formulations comprising triblocks of the present invention. Without wishing to be limited by a particular mode of administration, compositions or formulations of the invention may be delivered or administered to a subject (human or non-human) by any means known in the art. As none of the components making up a triblock of the invention is toxic to the human or animal subject, compositions or formulations of the invention may be formed in a form suitable for oral, aerosol, parenteral, subcutaneous, intravenous, intramuscular, interperitoneal, rectal or vaginal administrations.

In some embodiments, compositions or formulations of the invention are intended for administration by injection. The requirements for injectable compositions are well known to those of ordinary skill in the art. See Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

Triblocks of the invention are typically characterized by a liquid form at room temperature or around room temperature or below room temperature and a gel form at a temperature above room temperature or at a temperature that is around a physiological temperature. Thus, triblocks or compositions or formulation comprising same may be easily applied or delivered at room temperature to an object or to a target site. Upon delivery, at a higher temperature, the triblocks undergo solidification, resulting in a 3-dimensional matrix or scaffold which remains stable at the site to which it is delivered. In situ degradation of the triblock may depend on a multitude of parameters including, inert alia, the particular triblock used, the site of delivery, environmental conditions, and others.

Triblocks containing a cargo material, e.g., an active and/or a non-active agent, may release the cargo over time. While the release may be thermally controlled or induced, the release may alternatively proceed at a rate dependent on the natural in situ degradation of the triblock. Thus, independent on any external stimulus, triblocks of the invention may be used as vehicles for delivering at least one active or non-active agent to a target site; wherein delivery is achieved at the triblock liquid phase. The active agent and/or the non-active agent may be introduced into the triblock during preparation of the triblock or by dissolving the triblock in a medium permitting dissolution and homogeneous distribution of the cargo therein.

Thus, triblocks of the invention may be used for drug delivery, cell therapy, tissue engineering or in a variety of cosmetic applications.

For medicinal purposed, the triblock may be used with at least one active agent. The active agent may be any active drug used in the treatment or prophylaxis of a disease or disorder in a human or animal subject. For the sake of brevity, the multitude of drugs and medicaments that may be used as cargo in triblocks of the invention are not specified. Non-limiting examples of such drugs are included in https://www.drugs.com. The full list of drugs provided therein is herein incorporated by reference.

Non-limiting examples of active agents that may be used in triblocks of the invention include drugs and biologically active agents such as peptide and protein drugs, desensitizing agents, antigens, vaccine agents and vaccine antigens, anti-inflammatory agents including steroidal agents and non-steroidal agents, antibiotic agents, antimicrobial agents, anti-allergenic agents, anti-cholinergic agents, sedatives, tranquilizers, steroids, hormones, humoral agents, analgesics, anti-histamine agents, cardioactive agents, antiparkinsonian agents, antihypertensive agents, nutritional materials and others.

As the triblocks of the invention are biodegradable, they can act as drug delivery vehicles or as biodegradable compositions. Such may comprise a triblock copolymer of the invention and at least one active agent which may be selected fro human therapeutic use, human cosmetic use, animal veterinary use, agricultural use, for diagnostic or experimental use or any other active or non-active agent. Where veterinary compositions are concerned agents used in veterinary may be selected.

The drug delivery vehicles, namely the triblock, may be selected to permit release of the active or non-active agent therefrom over a predetermined or desired period of time. Release may be achieved via decomposition of the triblock, via its phase change, via induction of an external stimulus, e.g., thermal stimulus, via spontaneous release or any other means. Release may commence within several days after delivery and may proceed over a period of several days to several weeks, months or years.

Active or non-active agents may alternatively be used in cosmetics or for improving a subject's quality of life.

In some embodiments, for medicinal, cosmetic or any other use, the triblock of the invention may be used free of any active or non-active agents.

In some embodiments, the triblock, optionally comprising an active or a non-active agent, is used for tissue augmentation. A triblock composition of the invention may be used as a temporary filler in surgical medical situations as well as for cosmetic purposes, such as deep or shallow wrinkle filling. The presence of an active or a non-active agent will depend on the particular application and the application protocol of use.

In some embodiments, the triblock is used for cosmetic applications, wherein the triblock is not injected under the skin but rather applied onto a skin region of a subject. In such applications, a triblock composition may be tailored for application by a spray or a cream or by other topical means known in the art.

Triblocks and compositions or formulations comprising same may additionally be used in non-medical applications, e.g., agricultural, experimental or otherwise for constructing 3-dimensional objects. These may be shaped scaffolds, films, volume fillers and others.

In some embodiments, a triblock of the invention may be used as a matrix material for delivery of agents to a plant surface, e.g., by spraying, or for forming a coating or a protective layer on a plant surface.

Triblocks of the invention may further be used for the construction of 3D scaffolds by, e.g., 3D printing. In such applications, aqueous solutions of the triblock with or without additives or active agents may be printed at a temperature permitting material flow and subsequent solidification into a 3D scaffold. Such a scaffold may contain different active and/or non-active agents (e.g., RGD segments, vascularization inducers, nutrients, antibiotics, protein drugs and others), at different concentrations. For example, a scaffold printed from 3 polymers that liquefy at 18, 21 and 24° C., each loaded with a different agent, allow gradual elimination of scaffold network so that cooling to 23° C. liquefies the polymers of the 24° C. transition phase to form a loose scaffold. After another period when propagation of cells occurs, more space and less scaffolding is required. At this stage, the construct will be cooled to 20° C. to remove additional part of the polymer network and at the last stage, the growing tissue may be cooled to 17° C. for complete elimination of the polymer scaffold.

The invention further contemplates methods of treatment using triblocks of the invention, such methods comprising administering, e.g., by injection, a composition or a formulation comprising a triblock of the invention, optionally comprising at least one active agent, to a tissue of a subject.

Also provided are cosmetic methods using triblocks of the invention, such methods comprising administering, e.g., by injection or topically by a spray or a cream, a composition or a formulation comprising a triblock of the invention, optionally comprising at least one cosmetically active agent, to a skin region of a subject.

The invention further contemplates agricultural methods of using triblocks of the invention, such methods comprising delivering, e.g., by spraying or coating, a composition or a formulation comprising a triblock of the invention, optionally comprising at least one agriculturally active agent, to a surface region of a plant or an explant.

The invention thus contemplates:

A triblock copolymer constructed of a lactide-containing polyester and poly(ethylene glycol), PEG, wherein in the triblock copolymer, the PEG is of a molecular weight between 1,000 and 3,000 Da.

In some embodiments of all aspects of the invention, in a triblock copolymer according to the invention, the lactide-containing polyester is selected from D,L-PLA, L-PLA, D-PLA, PLGA and PLCL.

In some embodiments of all aspects of the invention, in a triblock copolymer according to the invention, the triblock having a structure selected from D,L-PLA-PEG-D,L-PLA, D-PLA-PEG-D-PLA, L-PLA-PEG-L-PLA, PLGA-PEG-PLGA and PCL-PEG-PCL.

In some embodiments of all aspects of the invention, in a triblock copolymer according to the invention, the triblock is selected from hybrid triblocks containing PEG and one lactide-containing polyester segment.

In some embodiments of all aspects of the invention, in a triblock copolymer according to the invention, the triblock being selected from D,L-PLA-PEG-L-PLA, D,L-PLA-PEG-D-PLA, D-PLA-PEG-L-PLA, L-PLA-PEG-PLGA, D-PLA-PEG-PLGA, PLGA-PEG-PCL and PCL-PEG-D,L-PLA.

In some embodiments of all aspects of the invention, in a triblock copolymer according to the invention, the lactide-containing polyester segment is PLGA.

In some embodiments of all aspects of the invention, in a triblock copolymer according to the invention, the triblock is PLGA-PEG-PLGA.

Also provided is a poly(D,L-lactic acid-co-glycolic acid)-b-poly(ethylene glycol)-b-poly (D,L-lactic acid-co-glycolic acid) (PLGA-PEG-PLGA) triblock copolymer, wherein in the PLGA-PEG-PLGA triblock copolymer, the PEG is of a molecular weight between 1,000 and 3,000 Da, the PLGA and PEG being present at a ratio of between 1 and 4, and wherein the triblock copolymer having a gelation temperature between 10 and 50° C.

Further provided is a PLGA-PEG-PLGA triblock copolymer, wherein in the PLGA-PEG-PLGA triblock copolymer, the PEG is of a molecular weight between 1,000 and 3,000 Da, one or both of the PLGA segments being constructed of lactide and glycolide (LA:GA) moieties at a ratio (LA:GA) of about 6, and wherein the triblock copolymer having a gelation temperature between 10 and 50° C.

Also provided is a PLGA-PEG-PLGA triblock, characterized by:
in PLGA-PEG-PLGA triblock copolymer, the PEG is of a molecular weight between 1,000 and 3,000 Da,
in the PLGA-PEG-PLGA triblock copolymer, the PLGA and PEG being present at a ratio of between 1 and 4, or one or both of the PLGA segments is constructed of lactide and glycolide (LA:GA) moieties at a ratio around about 6; and
the triblock copolymer having a gelation temperature between 10 and 50° C.

In some embodiments of all aspects of the invention, in a triblock copolymer according to the invention, the PLGA-PEG-PLGA triblock is characterized by:
in PLGA-PEG-PLGA triblock copolymer, the PEG is of a molecular weight between 1,000 and 3,000 Da,
in the PLGA-PEG-PLGA triblock copolymer, the PLGA and PEG being present at a ratio of between 1 and 4,
in the PLGA-PEG-PLGA triblock copolymer, one or both of the PLGA segments is constructed of lactide and glycolide (LA:GA) moieties at a ratio around about 6; and
the triblock copolymer having a gelation temperature between 10 and 50° C.

Also provided is a PLGA-PEG-PLGA triblock copolymer material, the material being any one or more of materials in Table 1.

Also provided is a method of manufacturing a PLGA-PEG-PLGA triblock copolymer having a gelation temperature between 10 and 50° C., the method comprising reacting PEG of a molecular weight between 1,000 and 3,000 Da with D,L-lactic acid (LA) and a glycolide (GA) at (1) a LA:GA ratio of about 6; and/or (2) with a D,L-lactic acid (LA) and a glycolide (GA) amounts sufficient to achieve a PLGA/PEG ratio of between 1 and 4; under conditions permitting formation of the triblock copolymer.

Also provided is a polymeric material comprising or consisting a triblock copolymer according to the invention.

In some embodiments of all aspects of the invention, in a triblock copolymer according to the invention, the PEG having a molecular weight between 1,000 and 1,100, 1,000 and 1,200, 1,000 and 1,300, 1,000 and 1,400, 1,000 and 1,500, 1,000 and 1,600, 1,000 and 1,700, 1,000 and 1,800, 1,000 and 1,900, 1,000 and 2,000, 1,000 and 2,100, 1,000 and 2,200, 1,000 and 2,300, 1,000 and 2,400, 1,000 and 2,500, 1,000 and 2,600, 1,000 and 2,700, 1,000 and 2,800, 1,000 and 2,900, 1,500 and 1,600, 1,500 and 1,700, 1,500 and 1,800, 1,500 and 1,900, 1,500 and 2,000, 1,500 and 2,100, 1,500 and 2,200, 1,500 and 2,300, 1,500 and 2,400, 1,500 and 2,500, 1,500 and 2,600, 1,500 and 2,700, 1,500 and 2,800 or between 1,500 and 2,900 Da.

In some embodiments of all aspects of the invention, in a triblock copolymer according to the invention, the PLGA:PEG ratio is 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.7, 3.8, 3.9 or 4.

In some embodiments of all aspects of the invention, in a triblock copolymer according to the invention, the PLGA:PEG ratio is 1.08, 1.19, 1.20, 1.25, 1.32, 1.34, 1.36, 1.37, 1.53, 1.58, 1.61, 1.66, 1.88, 1.89, 1.91, 1.95, 1.99, 2, 2.01, 2.12, 2.16, 2.18, 2.21, 2.34, 2.47 or 3.02.

In some embodiments of all aspects of the invention, in a triblock copolymer according to the invention, the triblock copolymer being prepared by polymerization of poly(ethylene glycol) (PEG) with D,L-lactide (LA) and glycolide (GA).

In some embodiments of all aspects of the invention, in a triblock copolymer according to the invention, the LA:GA MW ratio is between 5.4 and 6.6.

In some embodiments of all aspects of the invention, in a triblock copolymer according to the invention, the triblock copolymer being a liquid at a temperature below room temperature and a gel at a physiological temperature.

The invention also provides a formulation comprising at least one triblock copolymer according to the invention.

In some embodiments of all aspects of the invention, for a triblock copolymer according to the invention, the formulation further comprising a carrier.

In some embodiments of all aspects of the invention, for a triblock copolymer according to the invention, the formulation is in the form of an aqueous formulation or an aqueous gel.

In some embodiments of all aspects of the invention, for a triblock copolymer according to the invention, the formulation is an aqueous formulation comprising between 10 and 30% (W/V) of the at least one triblock copolymer.

In some embodiments of all aspects of the invention, for a triblock copolymer according to the invention, the formulation is adapted for oral, aerosol, parenteral, subcutaneous, intravenous, intramuscular, interperitoneal, rectal or vaginal administration.

In some embodiments of all aspects of the invention, for a triblock copolymer according to the invention, the formulation is in the form of a hydrogel comprising at least one triblock copolymer according to the invention.

In some embodiments of all aspects of the invention, for a triblock copolymer according to the invention, the formulation is in the form of a scaffold comprising at least one triblock copolymer according to the invention.

In some embodiments of all aspects of the invention, for a triblock copolymer according to the invention, the formulation is adapted for administration by injection.

In some embodiments of all aspects of the invention, for a triblock copolymer according to the invention, the formulation further comprising at least one active or non-active agent.

In some embodiments of all aspects of the invention, for a triblock copolymer according to the invention, the at least one active or non-active agent is contained within the triblock copolymer.

Also provided is a drug delivery vehicle comprising at least one active or non-active agent contained within a triblock copolymer according to the invention.

In some embodiments of all aspects of the invention, for a triblock copolymer according to the invention, the vehicle is for medicinal, cosmetic or veterinary use.

In some embodiments of all aspects of the invention, for a triblock copolymer according to the invention, the vehicle is adapted for release of the at least one active agent over a predetermined period of time.

In some embodiments of all aspects of the invention, for a triblock copolymer according to the invention, the vehicle is for use in tissue augmentation.

In some embodiments of all aspects of the invention, for a triblock copolymer according to the invention, the triblock is for use as a temporary filler in surgical medical situations.

A triblock copolymer or a vehicle according to the invention are provided for use as a cosmetic agent or formulation.

A triblock copolymer or a vehicle according to the invention are provided for use as a filling in deep or shallow wrinkles.

A method is provided for the treatment of at least one disease or disorder in a subject, the method comprising administering to the subject a triblock copolymer according to the invention or a formulation comprising same.

A cosmetic method is provided which comprises topically administering to a subject a triblock according to the invention or a formulation comprising same.

A thermoresponsive article is provided which comprises two or more triblock copolymers according to the invention, and at least one active or non-active agent, each of said two or more triblock copolymers liquefy at different temperatures, thereby permitting controlled release of said at least one active or non-active agent.

A controlled release article is provided which comprises two or more triblock copolymers according to the invention, and at least one active or non-active agent, each of said two or more triblock copolymers gel at a different temperature, and comprise a different active or non-active agent, thereby permitting release of said at least one active or non-active agent.

A biodegradable article is provide which comprises a triblock copolymer according to the invention.

In some embodiments of all aspects of the invention, for a triblock copolymer according to the invention, the article is selected from a suture, a film, a filler and a scaffold.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts transition from a solution to a gel upon heating from room temperature to physiological temperatures.

FIG. 2 provides the structure of a PLGA-PEG-PLGA triblock copolymer. In the formula, x represents the number of PEG repeating units, y represents LA and z represents GA. Without limitation, X=22-69; Y=7-56; Z=0-28, where Z<Y/2.

FIG. 3 provides a representative ¹H NMR spectrum of PLGA-PEG-PLGA triblock copolymer (16, Table 1) with peak assignments. LA:GA ratios were calculated by comparing the integration of their respective peaks (peak C represents the CH of lactide and D the CH₂ of glycolide), and overall polymer MW was determined by using a known integration of the PEG peak (A) and adding to it the total LA and GA content.

FIG. 4 provides a representative ¹³C NMR spectrum of PLGA-PEG-PLGA triblock copolymer (16, Table 1) with peak assignments. Peak B represents PLGA block ester bonds and peak F represents the ester bridge between PEG and PLGA blocks.

FIG. 5 provides a representative IR spectrum of PLGA-PEG-PLGA triblock copolymer (16, Table 1). A strong band at 1750 cm⁻¹ is observed for the formed polyester.

FIG. 6 provides a representative phase diagram of PLGA-PEG-PLGA (19, Table 1) aqueous solutions. As temperature increases the solution turns to a gel, and upon further heating a precipitate is formed.

FIG. 7 shows dependence of T_{gel} on PLGA/PEG ratio. For each set of polymers based on a particular PEG MW, a linear relationship has been defined between the polymer's aqueous gelling temperature in a 20% solution and the polymer structure's PLGA/PEG ratio.

FIG. 8 shows a release profile of paracetamol from hydrogel of PLGA-PEG-PLGA 13. Media was exchanged at 16, 40, and 64 h after gel was formed and paracetamol content in media was tracked by UV absorbance at 243 nm.

FIG. 9 shows a PLGA-PEG-PLGA triblock copolymer modified by (1) extending the PEG block and (2) employing PCL sidechains.

FIG. 10 depicts an exemplary use of triblocks of the invention for tissue engineering.

### DETAILED DESCRIPTION OF EMBODIMENTS

### Experimental

Materials

PEG-1000 was purchased from Union Carbide Chemicals and Plastics Company Inc. PEG-1500 was purchased from BDH Chemicals Ltd. PEG-2000 and stannous octoate were purchased from Sigma Aldrich. Lactide and glycolide were purchased from Purac Biochem Bv. Dichloromethane was purchased from Bio-Lab Ltd.

Synthesis

Lactide-based triblocks according to the invention, amongst them PLA-PEG-PLA and PLGA-PEG-PLGA triblock copolymers were prepared by ring-opening polymerization (ROP) of poly(ethylene glycol) (PEG) in the presence of stannous octoate catalyst.

A sample synthesis for the preparation of PLGA-PEG-PLGA is as follows:

50 μL of a 100 mg/mL solution of stannous octoate in dichloromethane (DCM) was added to a melt of PEG-1500 (595 mg, 0.397 mmol), D,L-lactide (696 mg, 4.83 mmol) and glycolide (94 mg, 0.81 mmol). Solvent was allowed to evaporate and the vial was purged with N₂. The mixture was stirred at 120° C. for 2 h, followed by overnight stirring at 150° C. The crude polymer was taken up in DCM and precipitated into ether to afford polymer 8 in quantitative yield. ¹H NMR (300 MHz, CDCl₃, δ): 5.22-5.14 (m, LA), 4.92-4.67 (m, GA), 3.64 (s, PEG), 1.56 (d, J=6 Hz, LA); ¹³C NMR (75 MHz, CDCl₃, δ): 169 (C═O), 166 (C═O), 72 (LA, CH), 70 (PEG, CH₂), 69 (PEG, CH₂), 66 (LA, CH), 64 (GA, CH₂), 61 (GA, CH₂), 16 (LA, CH₃); IR (NaCl): ν=1750 (s), 1452 (w), 1350 (w), 1184 (w), 1086 (s), 949 (w), 863 (w).

Nuclear Magnetic Resonance (NMR)

¹H and ¹³C NMR spectra were obtained on a Varian 300 MHz spectrometer with CDCl₃ as the solvent and tetramethylsilane as shift reference.

Infrared (IR) Spectroscopy

Infrared spectroscopy (2000 FTIR; PerkinElmer) was performed on polymer samples cast onto NaCl plates.

Determination of Gelling Temperature

20% w/v aqueous polymeric solutions were incubated at a given temperature for 10 minutes, and the vial was inverted to test for gelling. If the gel did not flow, the temperature was recorded as the gelling temperature of the solution (T_{gel}). Results are accurate to +/−2.0° C.

Release Study

Paracetamol was dissolved in 1 mL of 20% aqueous PLGA-PEG-PLGA solution at a ratio of 5:100 paracetamol:polymer (w/w). The solution was heated to 37° C. until gel was formed. 4 mL 0.1 M phosphate-buffered saline solution (PBS) was added on top of the gel at 37° C. Paracetamol released was measured by UV absorbance at 243 nm.

Results and Discussion

Lactide-Containing Polyester—PEG Triblock Copolymers

Poly(D,L-lactic acid)-poly(ethylene glycol)-poly(D,L-lactic acid) (PDLLA-PEG-PDLLA) triblock copolymers were synthesized by ring-opening polymerization (ROP) of D,L-lactide by PEG (MW 1500 Da) in the presence of stannous octoate. In one example, stannous octoate (50 μL of a 10% solution in dichloromethane) was added to a heated mixture of PEG-1500 (216.06 mg, 0.144 mmol) and D,L-lactide (410.37 mg, 2.84 mmol) under N₂ at 120° C. The mixture was stirred at 120° C. for 3 h, followed by overnight stirring at 150° C. to afford the polymer. A 20% w/v aqueous solution of the polymer formed a reversible thermoresponsive hydrogel with a sol-gel transition temperature of 40° C.

In another example, the same procedure was performed with PEG-1500 (297.9 mg, 0.199 mmol) and D,L-lactide (536.43, 3.72 mmol). A 20% w/v aqueous solution of the resultant polymer formed a reversible thermoresponsive hydrogel with a sol-gel transition temperature of 44° C.

Exemplary System 1: PLA-PEG-PLA Series

Poly(D,L-lactic acid)-poly(ethylene glycol)-poly(D,L-lactic acid) (PDLLA-PEG-PDLLA) triblock copolymers were synthesized by ring-opening polymerization (ROP) of D,L-lactide by PEG (MW 1500 Da) in the presence of stannous octoate. In one example, stannous octoate (50 μL of a 10% solution in dichloromethane) was added to a heated mixture of PEG-1500 (216.06 mg, 0.144 mmol) and D,L-lactide (410.37 mg, 2.84 mmol) under N₂ at 120° C. The mixture was stirred at 120° C. for 3 h, followed by overnight stirring at 150° C. to afford the polymer. A 20% w/v aqueous solution of the polymer formed a reversible thermoresponsive hydrogel with a sol-gel transition temperature of 40° C.

In another example, the same procedure was performed with PEG-1500 (297.9 mg, 0.199 mmol) and D,L-lactide (536.43, 3.72 mmol). A 20% w/v aqueous solution of the resultant polymer formed a reversible thermoresponsive hydrogel with a sol-gel transition temperature of 40° C.

Exemplary System 2: PLGA-PEG-PLGA Copolymer Series

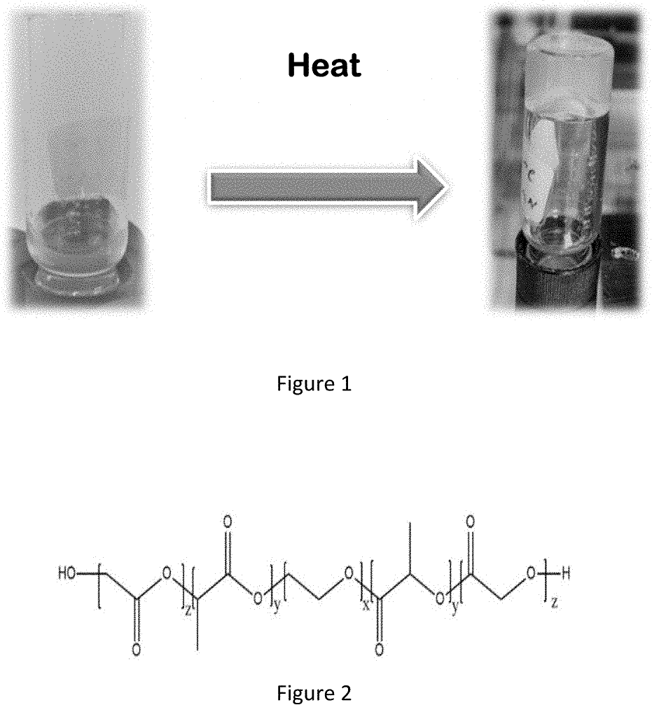

It has been demonstrated that the gelling temperature of aqueous solutions of PLGA-PEG-PLGA triblock copolymers was lowered by increasing either the lactide:glycolide (LA:GA) ratio in the PLGA block or polymer aqueous concentration. Here, a 6/1 LA:GA molar ratio and a 20% w/v aqueous polymer concentration were fixed in order to isolate the effect of PLGA/PEG weight ratio on gelling behaviour of the copolymer (FIG. 1). A series of such polymers (FIG. 2) were thereby synthesized with varying PEG and PLGA molecular weights and ratios, while keeping constant a 6:1 LA:GA molar ratio in the feed. Gelling temperature of 20% w/v aqueous solutions of each polymer were then tested (Table 1). Polymers with different molecular weight PLGA blocks were obtained by altering the ratio of combined LA and GA monomers relative to PEG in the feed.

In some embodiments of a triblock shown in FIG. 2, X=22-69; Y=7-56; Z=0-28 where Z<Y/2.

Characterization of PLGA-PEG-PLGA Copolymer Series

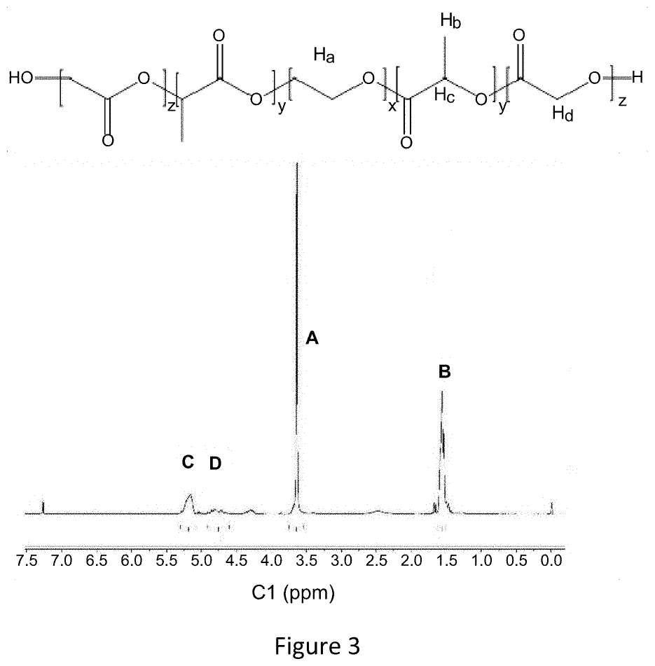

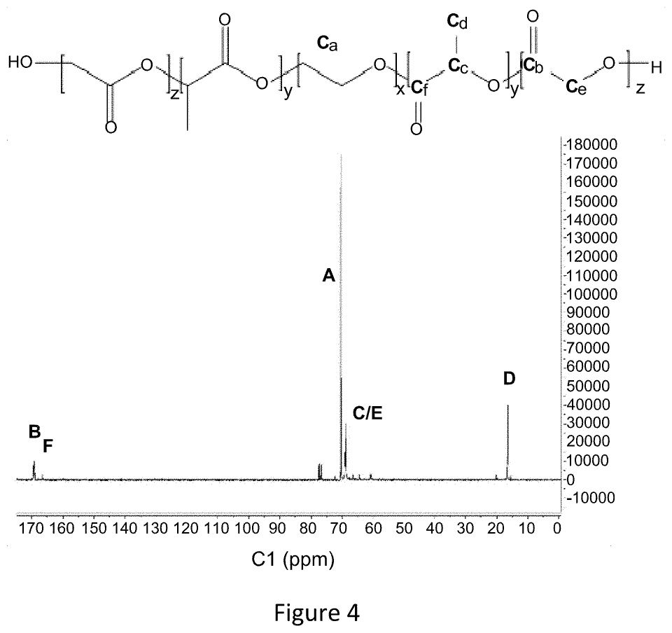

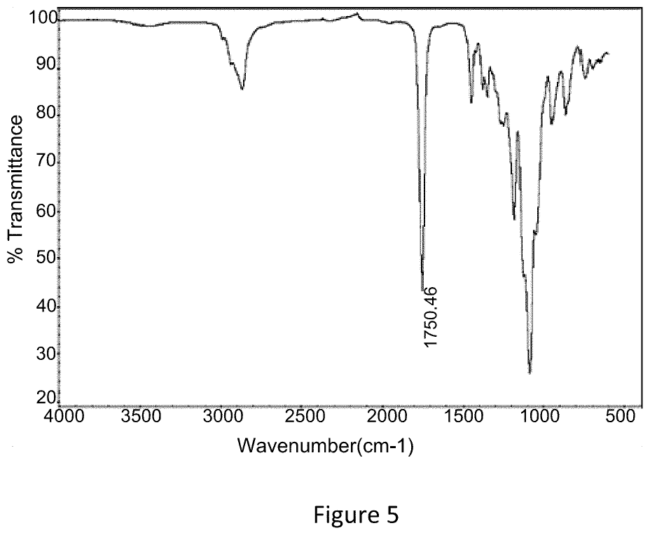

Polymer MW and experimental LA:GA ratio were defined by ¹H NMR (FIG. 3). To determine MW, the known integration value of the PEG peak (FIG. 3, peak A) was compared to the integrations of the lactide and glycolide peaks (FIG. 3, peaks C and D). The post-purification LA:GA ratio was also determined from the relative integration of the C (lactide CH) and D (glycolide CH₂) peaks of the ¹H NMR spectrum. Ester formation was confirmed by ¹³C NMR and IR. The carbon that experienced a chemical shift of 169 ppm corresponds to PLGA polyester and of 166 ppm to the ester connectivity between PEG and PLGA blocks (FIG. 4, peaks B and F). The IR ester band at 1750 cm⁻¹ also indicates ester bond formation (FIG. 5). The spectroscopy and gelling results for all 26 synthesized polymers can be found in Table 1.

TABLE 1
Chemical properties of PLGA-PEG-PLGA triblock copolymers.
Polymers 1-7 are based on PEG-1000, 8-20 on PEG-1500,
and 21-26 on PEG-2000. Polymers were synthesized by ROP
of D,L-lactide and glycolide by PEG in the presence of stannous
octoate catalyst. PLGA/PEG and LA:GA ratios and polymer MW were
determined by ¹H NMR (FIG. 3).

| | PEG | PLGA | PLGA/ | | T_{gel} |
|---|---|---|---|---|---|
| Entry | MW^{a} | MW^{b} | PEG^{c} | LA:GA^{d} | (C.) |
| 1 | 1000 | 1077 | 1.08 | 6.2 | 50 |
| 2 | 1000 | 1526 | 1.53 | 5.6 | 40 |
| 3 | 1000 | 1894 | 1.89 | 5.3 | 32 |
| 4 | 1000 | 1946 | 1.95 | 5.8 | 35 |
| 5 | 1000 | 2159 | 2.16 | 6.6 | 20 |
| 6 | 1000 | 2176 | 2.18 | 6.6 | 24 |
| 7 | 1000 | 2468 | 2.47 | 5.7 | 15 |
| 8 | 1500 | 1789 | 1.19 | 6.1 | 50 |
| 9 | 1500 | 1872 | 1.25 | 6.1 | 50 |
| 10 | 1500 | 2049 | 1.37 | 5.9 | 50 |
| 11 | 1500 | 2408 | 1.61 | 6.1 | 45 |
| 12 | 1500 | 2485 | 1.66 | 6.6 | 45 |
| 13 | 1500 | 2819 | 1.88 | 6.7 | 40 |
| 14 | 1500 | 2861 | 1.91 | 5.7 | 40 |
| 15 | 1500 | 2983 | 1.99 | 6.6 | 40 |
| 16 | 1500 | 3006 | 2.00 | 6.2 | 40 |
| 17 | 1500 | 3182 | 2.12 | 6.6 | 40 |
| 18 | 1500 | 3314 | 2.21 | 5.7 | 35 |
| 19 | 1500 | 3510 | 2.34 | 5.8 | 35 |
| 20 | 1500 | 4529 | 3.02 | 6.8 | 25 |
| 21 | 2000 | 2406 | 1.20 | 5.8 | 60 |
| 22 | 2000 | 2640 | 1.32 | 5.7 | 58 |
| 23 | 2000 | 2670 | 1.34 | 6.5 | 58 |
| 24 | 2000 | 2727 | 1.36 | 5.3 | 60 |
| 25 | 2000 | 3163 | 1.58 | 5.7 | 58 |
| 26 | 2000 | 4016 | 2.01 | 6.1 | 55 |

^{a}corresponds to X in the polymer structure (FIG. 1).
^{b}corresponds to Y + Z in the polymer structure (FIG. 1).
^{c}corresponds to (Y + Z)/X in the polymer structure (FIG. 1).
^{d}corresponds to Y/Z in the polymer structure (FIG. 1).

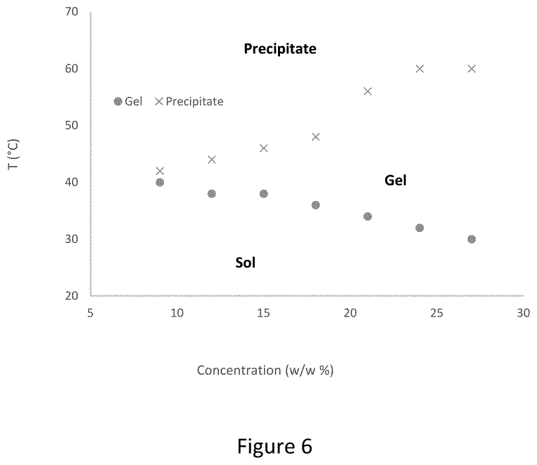

The mechanism of PLGA-PEG-PLGA triblock copolymer gel formation has been described. In short, at temperatures of about 0-25° C., the inter-chain hydrogen bonding between PEG segments dominates the solution energy profile, and the polymer dissolves in water. As the temperature increases, these hydrogen bonds weaken, and inter-chain hydrophobic interactions between the PLGA segments of the copolymer strengthen, leading to a three-dimensional physically cross-linked system that does not exclude water, resulting in the hydrogel. As the temperature is further increased, the hydrophobic interactions are further strengthened and the polymer crashes out of solution (FIG. 6). The PLGA/PEG ratio is therefore crucial in determining the sol-gel transition temperature (T_{gel}), as a low amount of hydrophobic inter-chain PLGA interactions relative to those of PEG would require a higher amount of energy to overcome the hydrophilic PEG-water and PEG-PEG interactions, and a high PLGA/PEG ratio would require less energy to overcome this barrier. Consequently, one would expect that a higher PLGA/PEG ratio would lead to a lower T_{gel}.

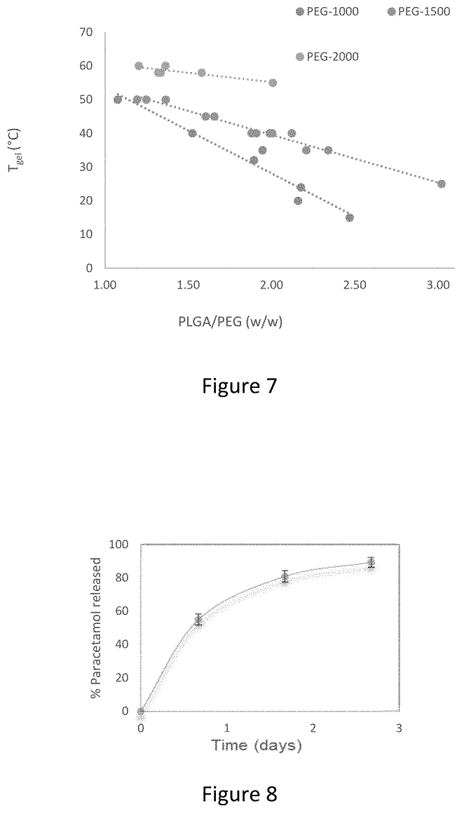

By controlling the LA:GA ratio, PEG molecular weight, and polymer concentration, we were able to isolate the effect of PLGA block length on gelling temperature. As expected, increase of hydrophobic PLGA block lead to a lower gelling temperature, as the system required less energy for the PLGA-PLGA hydrophobic interactions to overcome the hydrogen bonding between hydrophilic PEG segments. Indeed, a linear relationship was found between descending PLGA block length and gelling temperature (FIG. 7).

Controlled Release of a Representative Drug

The controlled release of paracetamol as representative water soluble drug from within the gel to external physiological media was tested to prove the ability of a therapeutic agent to be released from the gel matrix. To this effect, paracetamol was dissolved in an aqueous solution containing 20% w/v PLGA-PEG-PLGA 13, then heated to 37° C. to form gel. PBS was added on top of the gel, and it was exchanged for fresh PBS each morning until almost 90% of paracetamol had been observed in the exchanged media. We chose paracetamol as a representative drug as its release profile was easy to follow by UV absorbance of the exchanged media. Over 50% of the paracetamol was released within 16 hours, and 90% released within 64 hours (FIG. 8). It should be noted that the gel maintained its robustness in the release media for over one week with almost no erosion or change in viscosity. These results are consistent with previously reported water-soluble drug release from PLGA-PEG-PLGA thermoresponsive hydrogels.

Due to the robustness of the hydrogel, and its optimized sol-gel sharp transition, it can be injected into a physiological environment at room temperature as a liquid, and gel in tissue. When representative therapeutic material was dissolved in the room-temperature solution, controlled release from the gel was achieved. This finding may allow for the targeted delivery and controlled release of any therapeutic material at an injectable site.

Comparative Data

Where equivalent PCL-PEG-PCL triblocks were used instead of PLGA-PEG-PLGA, hydrogels were similarly obtained. In this case, PCL-PEG-PCL is an example that forms a non-reversible hydrogel.

Where in the PCL-PEG-PCL triblocks the PEG had a MW of 4000 polymers formed hydrogels only when the following two conditions were met:
i. PCL MW was in the range of 1,700-2,200 Da.
ii. The suspended polymer was heated to 50° C. for 10 min.

Water soluble solutions of this polymer did not form gels and remained in solution at any temperature up to ˜40° C. When heated at 50° C. these polymers forms gels that were not reversible. The gels remained stable at room temperature for over one month. Similar results were obtained for PCL-PEG(8000)-PCL when the PCL MW was in the range of 3,000-4,400. Although logically this triblock should form a reversible hydrogel, it did not; but only at a narrow MW range it formed a non-reversible gel.

When PEG of MW over 3,000 Da was used in PLGA-PEG-PLGA triblocks, gels were never formed. PEG 4,000 and PEG 8,000 triblock PLGA polymers were either water soluble or insoluble without a gelling phase.

Experimental—High Molecular Weight PCL-PEG-PCL Triblock Copolymers

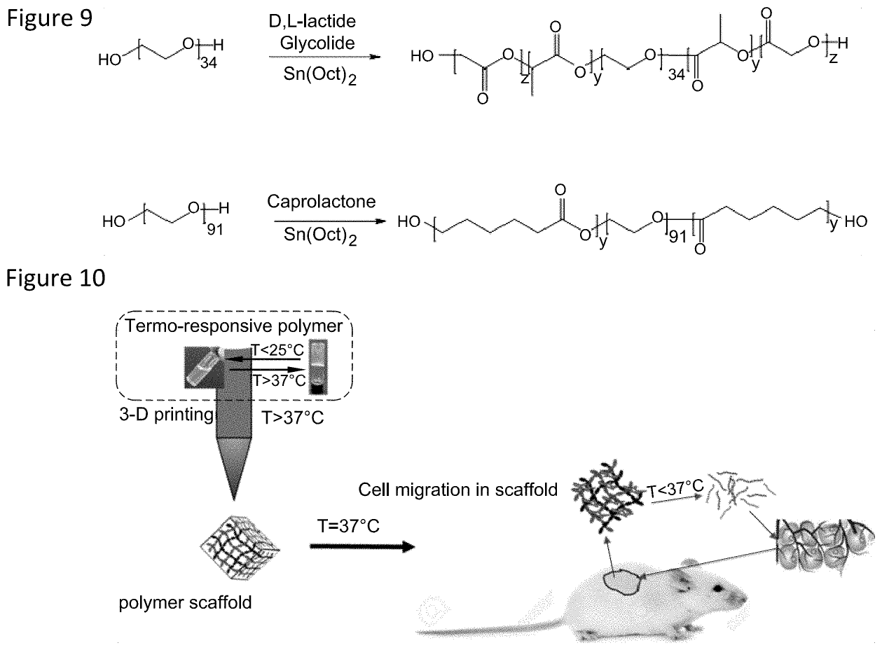

Hydrogel longevity and long-term stability may be enhanced by (1) replacing the PLGA sidechains with poly(caprolactone) (PCL), a more hydrophobic and hydrolytically stable polyester, and (2) using higher molecular weight (MW) PEG in order to afford higher MW biodegradable sidechains (FIG. 9).

The polymers were synthesized as follows, with relative amount of starting materials controlled to afford different MW PCL sidechains:

A 10% solution of stannous octoate (50 μL) was added to a melt of a 1:1 w/w mixture of PEG-4000 or PEG-8000 and ε-caprolactone under nitrogen atmosphere. The mixture was stirred at 120° C. for 2 h, followed by overnight stirring at 150° C.

Aqueous solutions of polymers were prepared at varying concentrations (10-30% w/w) and were tested for aqueous solubility and hydrogel stability. For a PEG-4000 starting material, PCL sidechains ranging from 1700-2200 afforded thermoresponsive hydrogels that formed gels upon heating to 50° C. Lower MW sidechains did not form gels up to 75° C., and higher MW sidechains were not dispersible in aqueous solution, even at 5% w/w. For triblock copolymers based on PEG-8000, a PCL MW range of 3000-3400 was shown to offer the same effect. In all cases, hydrogels were not reversible, so gel was maintained upon return to room temperature. Lower MW PEG-based polymers are ineffective for this application, as polymers thereof dissolve in aqueous solution and do not form gels. PLGA sidechains of PEG-4000 were either soluble (lower MW PLGA) or insoluble (as PLGA MW increased), and never formed gels.

Amongst the many possible applications, triblocks may be used for tissue engineering is illustrated in FIG. 10.

### Example: Mixture of Polymers for Reaching a Certain Gelling Temperature

PLGA-PEG-PLGA triblock copolymers that one gels at 42° C. 20% w/v solution in deionized water (DDW) (Polymer A) and the second triblock copolymer that forms a reversible hydrogel at 34° C. (Polymer B) were used in this study.

A solution of both polymers A and B was prepared by dissolving 200 mg of polymer A and 200 mg of polymer B in 2 mL of DDW, so that each polymer content is 10% w/v. The aqueous solution of polymers A and B was incubated at a given temperature for 10 min, and the vial was inverted to test for gelling. If the gel did not flow, the temperature was recorded as the gelling temperature of the solution (Tgel). Polymer blend A+B formed a reversible hydrogel at 38° C.

A blend of polymers A and B was prepared by dissolving 100 mg of polymer A and 300 mg of polymer B in 2 mL of DDW, so that total polymer concentration was 20% w/v with a 1:3 w/w ratio of A:B. The aqueous solution of blended A and B was incubated at a given temperature for 10 min, and the vial was inverted to test for gelling. Polymer blend A+B at 1:3 w/w formed a reversible hydrogel at 36° C.

A blend of polymers A and B was prepared by dissolving 300 mg of polymer A and 100 mg of polymer B in 2 mL of DDW, so that total polymer concentration was 20% w/v with a 3:1 w/w ratio of A:B. The polymer blend A+B 3:1 formed a reversible hydrogel at 40° C.

Similar mixtures of individual polymers that gel at different temperatures where prepared and showed an in between gelling temperature. The intermediate temperature was affected by the different polymers used as well as the relative w/w ratio of the polymers used to form the gel.

1. A triblock copolymer constructed of a lactide-containing polyester and poly(ethylene glycol), PEG, wherein in the triblock copolymer, the PEG is of a molecular weight between 1,000 and 3,000 Da.

2. The triblock copolymer according to claim 1, wherein the lactide-containing polyester is selected from D,L-PLA, L-PLA, D-PLA, PLGA and PCL.

3. The triblock copolymer according to claim 1, having a structure selected from D,L-PLA-PEG-D,L-PLA, D-PLA-PEG-D-PLA, L-PLA-PEG-L-PLA, PLGA-PEG-PLGA and PCL-PEG-PCL.

4. The triblock copolymer according to claim 1, being selected from hybrid triblocks containing PEG and one lactide-containing polyester segment.

5. The triblock copolymer according to claim 1, being selected from D,L-PLA-PEG-L-PLA, D,L-PLA-PEG-D-PLA, D-PLA-PEG-L-PLA, L-PLA-PEG-PLGA, D-PLA-PEG-PLGA, PLGA-PEG-PCL and PCL-PEG-D,L-PLA.

6. The triblock copolymer according to claim 1, wherein the lactide-containing polyester segment is PLGA.

7. The triblock copolymer according to claim 1, wherein the triblock is PLGA-PEG-PLGA.

8. A poly(D,L-lactic acid-co-glycolic acid)-b-poly(ethylene glycol)-b-poly (D,L-lactic acid-co-glycolic acid) (PLGA-PEG-PLGA) triblock copolymer, wherein in the PLGA-PEG-PLGA triblock copolymer, the PEG is of a molecular weight between 1,000 and 3,000 Da, the PLGA and PEG being present at a ratio of between 1 and 4, and wherein the triblock copolymer having a gelation temperature between 10 and 50° C.

9. A PLGA-PEG-PLGA triblock copolymer, wherein in the PLGA-PEG-PLGA triblock copolymer, the PEG is of a molecular weight between 1,000 and 3,000 Da, one or both of the PLGA segments being constructed of lactide and glycolide (LA:GA) moieties at a ratio (LA:GA) of about 6, and wherein the triblock copolymer having a gelation temperature between 10 and 50° C.

10. A PLGA-PEG-PLGA triblock, characterized by:
in PLGA-PEG-PLGA triblock copolymer, the PEG is of a molecular weight between 1,000 and 3,000 Da,
in the PLGA-PEG-PLGA triblock copolymer, the PLGA and PEG being present at a ratio of between 1 and 4, or one or both of the PLGA segments is constructed of lactide and glycolide (LA:GA) moieties at a ratio around about 6; and
the triblock copolymer having a gelation temperature between 10 and 50° C.

11. The PLGA-PEG-PLGA triblock according to claim 8, characterized by:
in PLGA-PEG-PLGA triblock copolymer, the PEG is of a molecular weight between 1,000 and 3,000 Da,
in the PLGA-PEG-PLGA triblock copolymer, the PLGA and PEG being present at a ratio of between 1 and 4,
in the PLGA-PEG-PLGA triblock copolymer, one or both of the PLGA segments is constructed of lactide and glycolide (LA:GA) moieties at a ratio around about 6; and
the triblock copolymer having a gelation temperature between 10 and 50° C.

12. A PLGA-PEG-PLGA triblock copolymer material, the material being any one or more of materials in Table 1.

13. A method of manufacturing a PLGA-PEG-PLGA triblock copolymer having a gelation temperature between 10 and 50° C., the method comprising reacting PEG of a molecular weight between 1,000 and 3,000 Da with D,L-lactic acid (LA) and a glycolide (GA) at (1) a LA:GA ratio of about 6; and/or (2) with a D,L-lactic acid (LA) and a glycolide (GA) amounts sufficient to achieve a PLGA/PEG ratio of between 1 and 4; under conditions permitting formation of the triblock copolymer.

14.-30. (canceled)

31. A drug delivery vehicle comprising at least one active or non-active agent contained within a triblock copolymer according to claim 1.

32. The vehicle according to claim 31, for medicinal, cosmetic or veterinary use.

33. The vehicle according to claim 31, adapted for release of the at least one active agent over a predetermined period of time.

34.-37. (canceled)

38. A method of treatment of at least one disease or disorder in a subject, the method comprising administering to the subject a triblock copolymer according to claim 1 or a formulation comprising same.

39. A cosmetic method comprising topically administering to a subject a triblock according to claim 1 or a formulation comprising same.

40. A thermoresponsive article comprising two or more triblock copolymers according to claim 1, and at least one active or non-active agent, each of said two or more triblock copolymers liquefy at different temperatures, thereby permitting controlled release of said at least one active or non-active agent.

41. A controlled release article comprising two or more triblock copolymers according to claim 1, and at least one active or non-active agent, each of said two or more triblock copolymers gel at a different temperature, and comprise a different active or non-active agent, thereby permitting release of said at least one active or non-active agent.

42.-43. (canceled)
